(19) United States
(12) Patent Application Publication
(10) Pub. No.: US 2015/0045613 A1
(43) Pub. Date: Feb. 12, 2015

(54) Malleable Surgical Assistance Devices

(71) Applicant: Norton EDWARDS, Nashville, TN (US)

(72) Inventor: Norton EDWARDS, Nashville, TN (US)

(21) Appl. No.: 14/279,473

(22) Filed: May 16, 2014

Related U.S. Application Data
(60) Provisional application No. 61/824,325, filed on May 16, 2013.

(51) Int. Cl. A61B 1/00; A61B 1/015; A61B 1/005

(52) U.S. Cl. CPC A61B 1/00147; A61B 1/00096; A61B 1/005; A61B 1/015
USPC 600/102

(57) ABSTRACT

An array of surgical assistance devices that may be used to provide a source of light to the surgical site; collect video or photo imaging of the surgical site; and/or provide smoke/vapor evacuation from the surgical site. The primary elements of the invention include lengths of flexible or malleable tubing or light wave guides that, through a first flexible section, may be connected to standard operating room equipment, through to a moveable base that may be positioned by the surgeon or the surgery personnel. The moveable base includes a clip structure designed to receive the tubing or wave guide conduit, and to be placed on or near the patient at or near the surgical site. The base may be provided with a moderately weighted material to facilitate the support of the surgical assistance device or may comprise a bendable ribbon or band with a securing clip. Extending from the moveable base is a malleable or bendable section of the tubing or wave guide that extends toward the surgical site to a terminal point configured with an appropriate light output, imaging lens, or evacuation flute. Additional components within the overall system facilitate the use of the devices by the surgeon for both the active surgical process and as a record keeping function of the same.

### CROSS REFERENCES TO RELATED APPLICATIONS

This application claims the benefit under Title 35 United States Code §119(e) of U.S. Provisional Application 61/824,325, filed May 16, 2013, the full disclosure of which is incorporated herein by reference.

### BACKGROUND OF THE INVENTION

1. Field of the Invention

The present invention relates generally to devices used in surgical operating room environments to facilitate the surgical process. The present invention relates more specifically to an array of surgical assistance devices comprising malleable (bendable) conduits and tubes used for surgical site illumination, imaging of the surgical site, and evacuation of smoke and vapor from the surgical site.

2. Description of the Related Art

Various complex systems are presently available to provide illumination of a surgical site, video imaging of a surgical site, and smoke or vapor evacuation from a surgical site. Most all of these systems, however, are extremely complex, costly, and frequently include components that must be disposed of after use. The complexity of such systems generally contributes to their difficulty of use, being cumbersome to position and re-position as required. Most such systems rely on large, bulky, and cumbersome brackets and frames to position devices near the surgical site without interfering with the access of the surgeon to the operation. Lights, video equipment, and evacuation devices are currently configured on articulating arms and the like that extend over and down to the operating table (from ceiling, wall, or floor mounted brackets) in a manner that is difficult for the surgeon to manipulate. Most such systems additionally involve terminal structures that, due to their complexity, cannot be easily sanitized and reused.

It would be desirable if a surgeon had at his or her disposal much simpler but equally functional devices that could be easily manipulated at the surgical site and could further be easily sanitized after use so as to be re-used in subsequent surgeries. It would be desirable if such components were lightweight yet easily positionable in a stable arrangement in conjunction with the surgical site. It would be desirable if such devices could connect to standard equipment utilized for light sources, video imaging equipment, and smoke/vapor evacuation (negative pressure) systems.

### SUMMARY OF THE INVENTION

The present invention provides an array of surgical assistance devices that may be used to provide a source of light to the surgical site; collect video or photo imaging of the surgical site, and/or provide smoke/vapor evacuation from the surgical site. The primary elements of the present invention include lengths of flexible or malleable tubing or light wave guides that, through a first flexible section, may be connected to standard operating room equipment, through to a moveable base that may be positioned by the surgeon or the surgical assistance. The moveable base includes a clip structure designed to receive the tubing or wave guide conduit, and to be placed on or near the patient at or near the surgical site. The base may be provided with a moderately weighted material to facilitate the support of the surgical assistance device. Extending from the moveable base is a malleable or bendable section of the tubing or wave guide that extends toward the surgical site to a terminal point configured with an appropriate light output, imaging lens, or evacuation flute. Additional components within the overall system facilitate the use of the individual devices by the surgeon for both the active surgical process and a record keeping function of the same.

### BRIEF DESCRIPTION OF THE DRAWINGS

FIG. 1 is a perspective view of the malleable light source device of the present invention shown positioned on a support base next to a surgical site.

FIG. 2 is a partially schematic top plan view of the light source device of the present invention showing both the flexible section and the bendable section.

FIGS. 3A & 3B are side and cross-sectional views of the malleable wave guide section for a light source of the present invention showing a three wire configuration.

FIGS. 4A & 4B are side and cross-sectional views of the malleable wave guide section for a light source of the present invention showing a two wire configuration.

FIGS. 5A & 5B are side and cross sectional views of the malleable wave guide section for a light source of the present invention showing a helix wall tube configuration.

FIGS. 6A & 6B are side and cross-sectional views of the malleable wave guide section for a light source of the present invention showing a bendable joint configuration.

FIG. 7 is an end view of the fiber wave guide light output section of the light source device of the present invention.

FIGS. 8A & 8B are end and side views of the smoke/vapor evacuation flute structure of the present invention.

FIG. 9 is a top plan view of a bilateral splitter structure capable of functioning in conjunction with either the light source device or the smoke/vapor evacuation device of the present invention.

FIG. 10 is a top plan view showing implementation of the bilateral splitter structure of the present invention utilized in conjunction with two light sources positioned near the surgical site.

FIG. 11 is a schematic block diagram showing the basic components of the light source device of the system of the present invention.

FIG. 12 is a schematic block diagram showing the basic components of the smoke/vapor evacuation device of the system of the present invention.

FIG. 13 is a schematic block diagram showing the basic components of the video/photo imaging device of the system of the present invention.

FIG. 14 is a schematic block diagram showing the implementation of both an imaging device of the present invention and a light source device of the present invention, in a typical operating room environment.

FIG. 15 is a top plan view of an alternate embodiment of the support base component of the present invention.

### DETAILED DESCRIPTION OF THE PREFERRED EMBODIMENTS

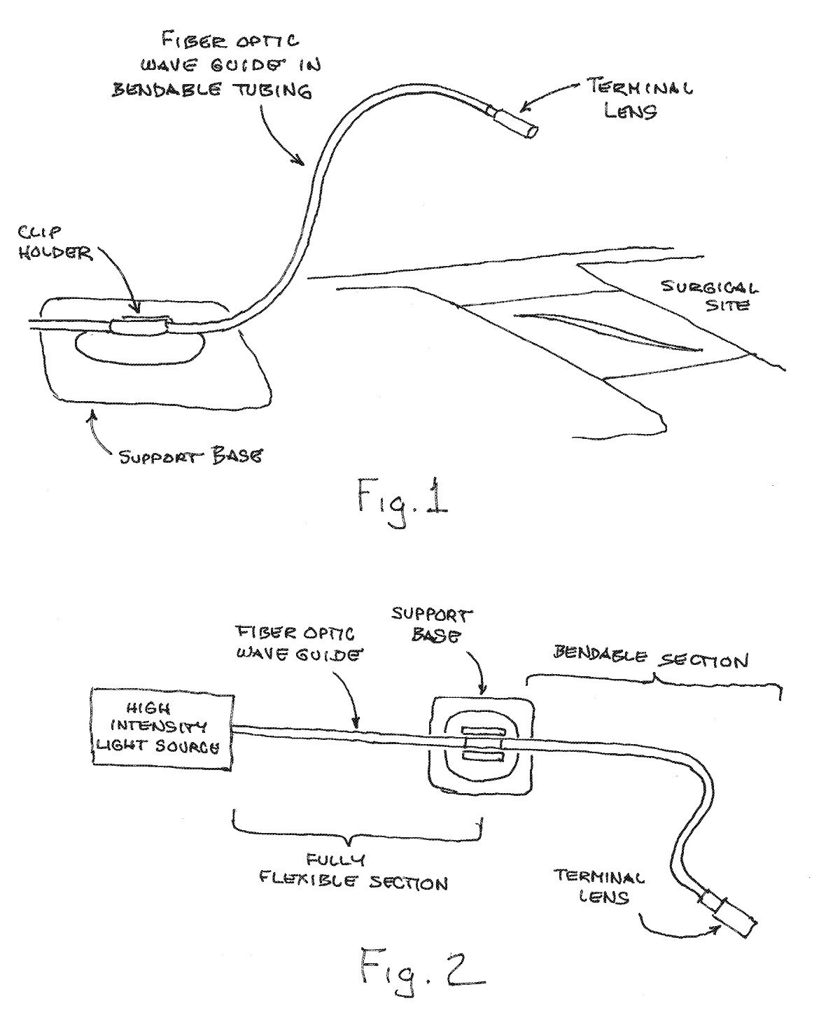

FIG. 1 shows the basic components of the system of the present invention (each of the various devices described) including a support base incorporating a clip holder structured to receive and hold a section of tubing or wave guide. The important part of the tubing or wave guide (fiber optic wave guide in the case of the light device shown in FIG. 1) is the malleable or bendable section that extends from the support base to a terminal point positioned over or near the surgical site. In the case of the light source, this terminal end comprises an output lens that may columnate or spread the light as needed. Alternate embodiments of the invention may include a lens structure on the terminal of the malleable wave guide that allows the surgeon to adjust the focus of the light source so as to spread the light or focus it in a beam.

FIG. 2 shows the various sections of the fiber optic wave guide as it extends from a high intensity light source such as is typically used in operating room environments. A first fully flexible section extends from the high intensity light source to the support base. At the point where the wave guide clips into the support base, a change in the structure of the shield or shroud around the wave guide occurs. From the support base to the terminal lens, the wave guide is integrated into a bendable section as described in more detail below.

FIGS. 3A & 3B provide a first example of a structure for the wave guide that incorporates a fiber optic bundle surrounded by a standard casing, which itself is surrounded by one or more spiraling bendable/malleable wires. This assembly is then enclosed within a sheath that may be tightly adhered to the assembly so as to prevent the bendable/malleable wires from being exposed externally. FIG. 3B shows a cross-sectional view of this structure along section line A-A′ in FIG. 3A.

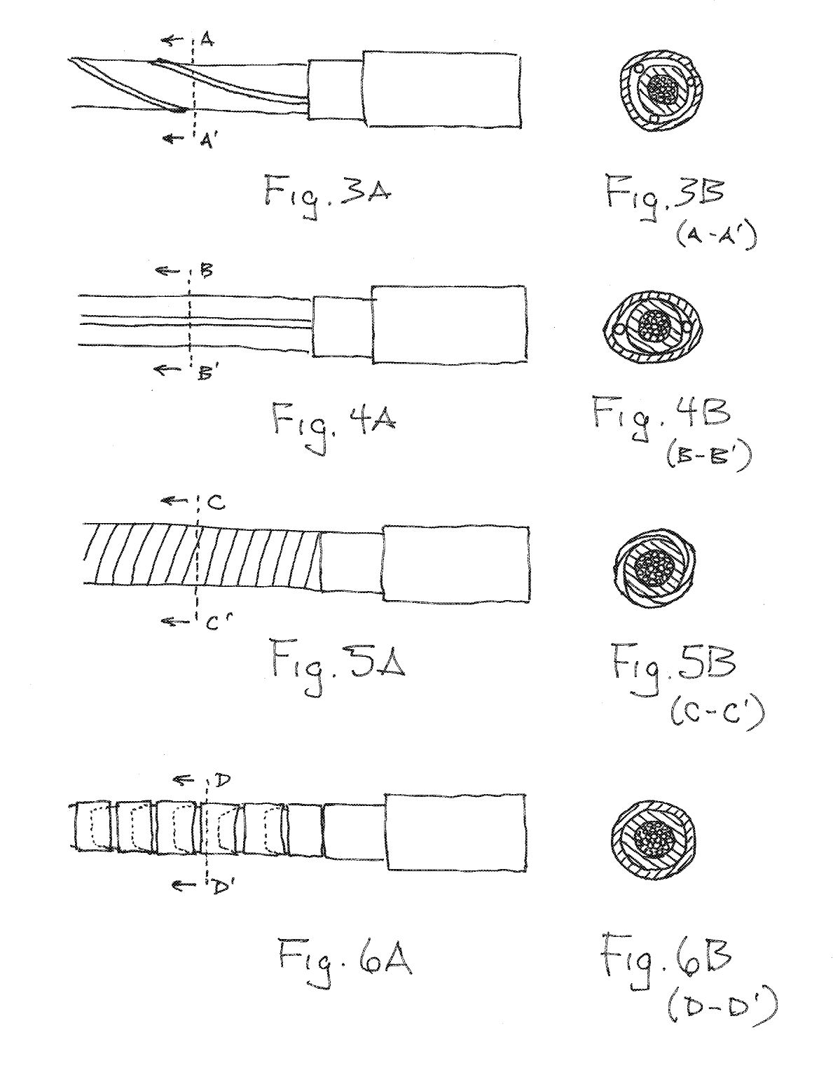

FIGS. 4A & 4B provide an alternate embodiment for the malleable/bendable structure comprising two wires that may extend in parallel along opposing sides of the encased fiber optic wave guide. Here as well, a shroud or sheath extends over the entire assembly so as to enclose the wires and wave guide. FIG. 4B shows the cross-sectional view seen at section line B-B′ in FIG. 4A.

FIGS. 5A & 5B provide yet another embodiment of the structure of the present invention wherein a helix conduit is positioned around the fiber optic wave guide. The frictional interaction of the helix coiled wall conduit provides a stiffness to the sheath that allows the overall assembly to be malleable/bendable. FIG. 5B shows the cross-sectional view of this structure at section line C-C′ in FIG. 5A.

FIGS. 6A & 6B provide a further alternate embodiment for the bendable/malleable structure of the present invention involving a plurality of joints comprising rigid cylinders with tapered connectors that collectively permit the bending of the otherwise rigid sheath around the fiber optic cable.

It is understood that although the above description of the various embodiments has been made in conjunction with the light source wave guide device of the present invention, both the video/photo imaging device of the present invention, and the smoke/vapor evacuation device of the present invention, may utilize the same or similar bendable sheath constructions.

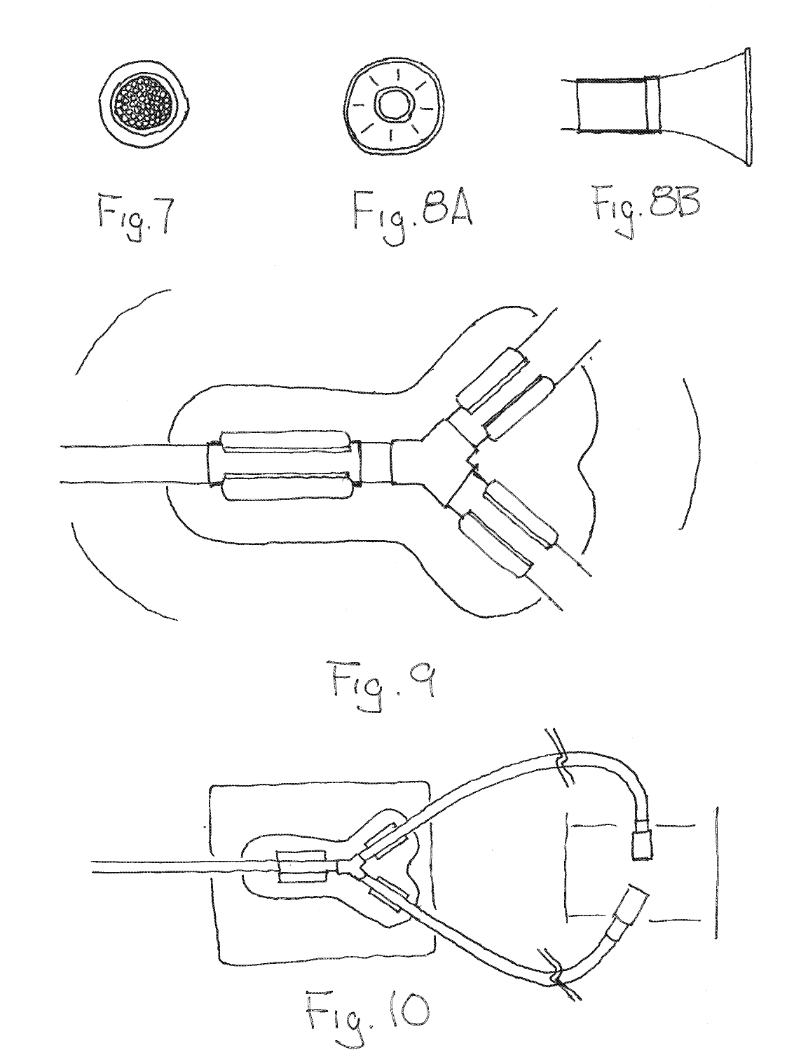

FIG. 7 shows the basic structure required at the end of the light source device of the present invention involving the terminal end of a fiber optic bundle surrounded by a bundle sheath and further surrounded by a rigid cylindrical end shroud.

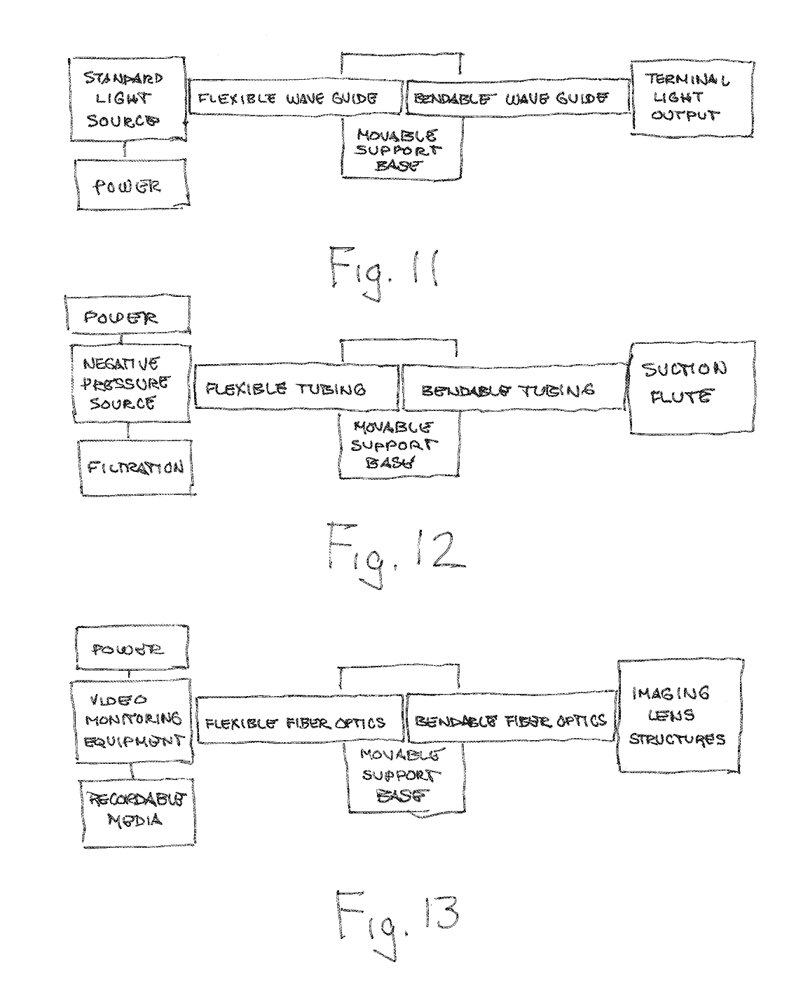

FIGS. 8A & 8B provide end and side views of the smoke/vapor suction flute that is preferably positioned at the end of an open tubing section for the evacuation device of the present invention. The smoke/vapor evacuation device would simply replace the fiber optic bundle of the light source device with an open section of tubing. Again, this would be structured first within a bendable/malleable section and then, after passing through the clipped-in portion of the support base, a flexible length of tubing to the negative pressure source.

FIG. 9 shows the manner in which the light source and smoke/vapor evacuation devices of the present invention may be bifurcated through a Y-connector positioned on the support base. Clips for the single incoming flexible section, as well as clips for the two outgoing malleable/bendable sections are shown.

FIG. 10 discloses the use of a bifurcated base and clip arrangement as that described in FIG. 9, providing in this example two separate light sources positionable over the surgical site. Similar implementation of the smoke/vapor evacuation device may be made.

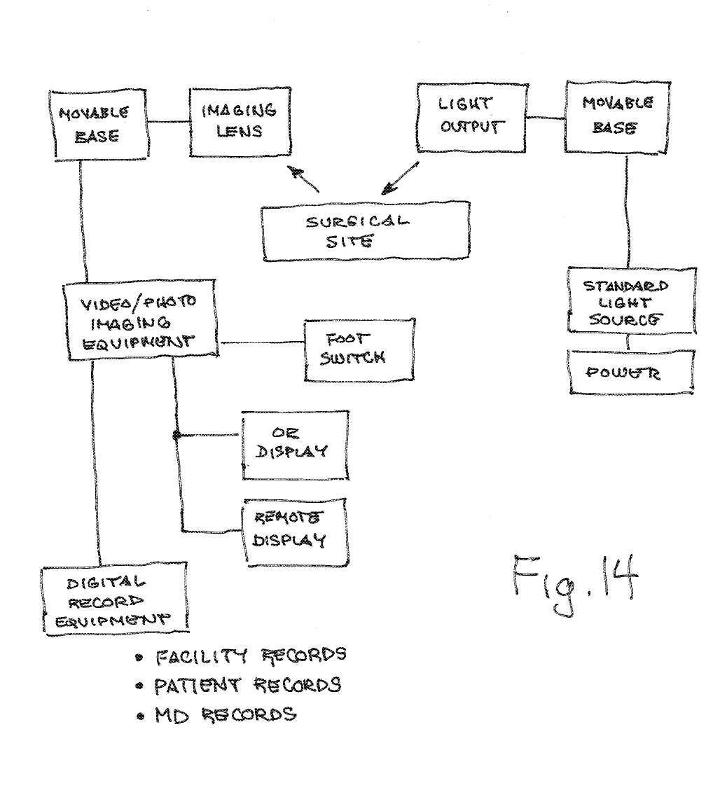

Reference is next made to FIGS. 11-13 which are schematic block diagrams showing the basic components necessary for implementation of each of the devices of the system of the present invention. FIG. 11 shows that the light source device of the present invention is made up of a flexible wave guide section as well as a bendable wave guide section which are preferably interfaced on the movable support base component. Terminal light output is provided at the end of the bendable wave guide section. Standard light source instrumentation is provided at the input to the flexible wave guide section. Power as necessary is provided to the standard light source instrumentation.

FIG. 12 shows a similar arrangement for the smoke/vapor evacuation device which comprises a flexible tubing section and a bendable tubing section, again preferably interfaced at the movable support base. A suction flute is provided at the input to the bendable tubing section and connection to a negative pressure source is provided at the output of the flexible tubing section. Necessary power connection is provided to the negative pressure source as well as filtration equipment provided at the output from the device to the negative pressure source.

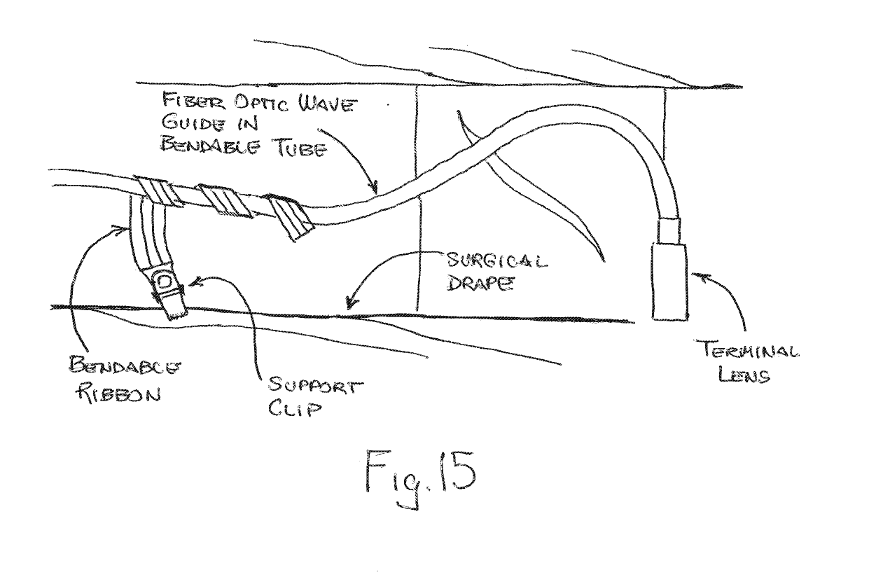

FIG. 13 discloses the basic components necessary for the video/photo imaging device of the present invention and includes a flexible fiber optic section as well as a bendable fiber optic section, again interfaced at the movable support base. Once again, it is understood that the movable support base preferably does not provide a physical interface between the flexible and bendable sections of each device, but merely provides the best location for terminating the structures of the system that provide for the malleable/bendable characteristics of the operational end of the device. That is, the preference is to have a continuous internal wave guide or tubing section extending all the way from a point over the surgical site to the standard operating room equipment operable in conjunction with the device. The movable support base and its clip-in configuration provide the best location for beginning the bendable/malleable part of the tubing or wave guide.

Additionally shown in FIG. 13 in conjunction with the video monitoring equipment, which receives the optical image through the flexible fiber optics in the system, are the necessary power source as well as preferably recordable media instrumentation.

Reference is next made to FIG. 14 which shows one possible implementation of the devices of the system of the present invention utilized in conjunction with a surgical site that includes both the use of the light source device, and the video/photo imaging device. Those skilled in the art will recognize that an additional use of the smoke/vapor evacuation device of the present invention could be incorporated into the surgical site arrangement as well.

FIG. 14 shows an implementation of the imaging system in conjunction with the imaging lens and its movable base connected to standard video photo imaging equipment. Modifications to this equipment could include a foot switch operable by the surgeon to initiate or terminate the video/photo imaging function. The overall video/photo imaging equipment system would be preferably connected to an operating room display (as is partially known in the art) and further to remote displays to provide close-up imaging of the surgery.

More importantly, the simplicity of the video imaging component of the system of the present invention lends itself to providing a simple and efficient manner of creating a digital record of the surgery, either in a continuous video of the surgery, or through the use of intermittent photo images created by the surgeon, such as through operation of the foot switch. Such a digital record, carried out by digital recording equipment as shown connected to the video/photo imaging equipment, might provide appropriate facility records, patient records, or physician records.

Reference is finally made to FIG. 15 which discloses an alternate support base structure comprising a bendable ribbon or band that may wrapped around a portion of the wave guide, tubing, or cable, and clipped to a secured section of surgical drape typically used to surround the surgical site.

The present invention therefore provides a basic structure embodied within a number of specific tools that include:

(A) Malleable Light Cable.

(B) Malleable Video/Camera Cable.

(C) Malleable Smoke Evacuation Tubing.

(D) Malleable LED Lay Down Strip—A malleable strip of LED lights which physicians and nurses may place down inside the body cavity to view the surgical sight better.

Each of these devices (except for the LED strip) would have a distal 2-3 foot malleable section and a proximal 6-8 feet made of typical non-malleable material. As well as the distal third being malleable, there is also a silicone pad that may secure the devices so they would not move around. As used in the present disclosure, the terms distal and proximal reference the surgical instrumentation to which the devices connect (as opposed to the surgical sight or the patient).

Although the present invention is described in conjunction with various preferred embodiments, those skilled in the art will recognize further implementations of the basic structures of the devices of the present invention with additional alternate embodiments. Various other structures for creating the malleable/bendable section of the tubing and/or wave guide component of the system are anticipated. The key characteristics of the components of the system include their simplicity, ruggedness, and ability to be sanitized and re-used. The quality of the fiber optic wave guide components should be determined by what would be suitable for use as a light source (lower quality wave guide characteristics) or as an imaging system (higher quality wave guide characteristics). The structures of the base component likewise may vary as the basic requirements of providing a moveable support that is not too heavy to be placed on or near the patient during surgery may be met by a number of different structures and materials. Various connectors between the devices of the present invention and the standard operating equipment with which they are intended to operate are also anticipated.

I claim:

1. A surgical assistance device for improving visual access to a surgical sight, the surgical assistance device comprising:
a first length of tubing comprising a flexible but non-malleable conduit, the first length of tubing having a proximal and a distal end, the proximal end connectable to standard operating room equipment;
a second length of tubing comprising a flexible malleable conduit, the second length of tubing having a proximal and a distal end, the proximal end connected to the distal end of the first length of tubing;
a terminal visual improvement instrument positioned on the distal end of the second length of tubing and oriented to operate to improve visual access to the surgical sight; and
a moveable base positioned at or near the connection between the first and second lengths of tubing, the moveable base comprising a clip structure to receive and secure one or both of the lengths of tubing.
